SEQUENCE LISTING (133 sequences)

SEQ ID NO: 1 - DNA, 52 bp, Artificial Sequence
  Description of Artificial Sequence Synthetic oligonucleotide
agagagagagagagagagagagagagagagagagagagagagagagagagag

SEQ ID NO: 2 - DNA, 30 bp, Artificial Sequence
  Description of Artificial Sequence Synthetic oligonucleotide
gagagagagagagagagagagagagagaga

SEQ ID NO: 3 - DNA, 20 bp, Artificial Sequence
  Description of Artificial Sequence Synthetic oligonucleotide
gatggatggatggatggatg

SEQ ID NO: 4 - DNA, 34 bp, Artificial Sequence
  Description of Artificial Sequence Synthetic oligonucleotide
gagagagagagagagagagagagagagagagaga

SEQ ID NO: 5 - DNA, 60 bp, Artificial Sequence
  Description of Artificial Sequence Synthetic oligonucleotide
gagagagagagagagagagagagagagagagagagagagagagagagagagagagagaga

SEQ ID NO: 6 - DNA, 48 bp, Artificial Sequence
  Description of Artificial Sequence Synthetic oligonucleotide
gagagagagagagagagagagagagagagagagagagagagagagaga

SEQ ID NO: 7 - DNA, 62 bp, Artificial Sequence
  Description of Artificial Sequence Synthetic oligonucleotide
gagagagagagagagagagagagagagagagagagagagagagagagagagagagagagaga

SEQ ID NO: 8 - DNA, 68 bp, Artificial Sequence
  Description of Artificial Sequence Synthetic oligonucleotide
gagagagagagagagagagagagagagagagagagagagagagagagagagagagagagagagagaga

SEQ ID NO: 9 - DNA, 30 bp, Artificial Sequence; identical to SEQ ID NO: 2
  Description of Artificial Sequence Synthetic oligonucleotide

SEQ ID NO: 10 - DNA, 42 bp, Artificial Sequence
  Description of Artificial Sequence Synthetic oligonucleotide
cttcttcttcttcttcttcttcttcctcttcttcttcttctt

SEQ ID NO: 11 - DNA, 21 bp, Artificial Sequence
  Description of Artificial Sequence Synthetic oligonucleotide
cagcagcagcagcagcagcag

SEQ ID NO: 12 - DNA, 32 bp, Artificial Sequence
  Description of Artificial Sequence Synthetic oligonucleotide
gagagagagagagagagagagagagagagaga

SEQ ID NO: 13 - DNA, 66 bp, Artificial Sequence
  Description of Artificial Sequence Synthetic oligonucleotide
cttcttcttcttcttcttcttcttcttcttcttcttcttcttcttcttcttcttcttcttcttctt

SEQ ID NO: 14 - DNA, 36 bp, Artificial Sequence
  Description of Artificial Sequence Synthetic oligonucleotide
aagaagaagaagaagaagaagaagaagaagaagaag

SEQ ID NO: 15 - DNA, 34 bp, Artificial Sequence; identical to SEQ ID NO: 4
  Description of Artificial Sequence Synthetic oligonucleotide

SEQ ID NO: 16 - DNA, 61 bp, Artificial Sequence
  Description of Artificial Sequence Synthetic oligonucleotide
tattattattattatcattattattattattattattattattattattattattattatt

SEQ ID NO: 17 - DNA, 52 bp, Artificial Sequence
  Description of Artificial Sequence Synthetic oligonucleotide
gagagagagagagagagagagagagagagagagagagagagagagagagaga

SEQ ID NO: 18 - DNA, 21 bp, Artificial Sequence
  Description of Artificial Sequence Synthetic primer
aacgcgaggacacgtacttac

SEQ ID NO: 19 - DNA, 20 bp, Artificial Sequence
  Description of Artificial Sequence Synthetic primer
ccgtcgccgtagtagagaag

SEQ ID NO: 20 - DNA, 22 bp, Artificial Sequence
  Description of Artificial Sequence Synthetic primer
cagattggagatgaagtcctcc

SEQ ID NO: 21 - DNA, 19 bp, Artificial Sequence
  Description of Artificial Sequence Synthetic primer
ccggcgataaaacaatgag

SEQ ID NO: 22 - DNA, 20 bp, Artificial Sequence
  Description of Artificial Sequence Synthetic primer
ccgctactaatagcagagag

SEQ ID NO: 23 - DNA, 20 bp, Artificial Sequence
  Description of Artificial Sequence Synthetic primer
gagccaaataagatcgctga

SEQ ID NO: 24 - DNA, 19 bp, Artificial Sequence
  Description of Artificial Sequence Synthetic primer
acgggcaatccgaacaacc

SEQ ID NO: 25 - DNA, 20 bp, Artificial Sequence
  Description of Artificial Sequence Synthetic primer
gcgaaaacacaatgcaaaaa

SEQ ID NO: 26 - DNA, 20 bp, Artificial Sequence
  Description of Artificial Sequence Synthetic primer
catgcaacaacgtcaccttc

SEQ ID NO: 27 - DNA, 20 bp, Artificial Sequence
  Description of Artificial Sequence Synthetic primer
ggcccgtccaagaaatattg

SEQ ID NO: 28 - DNA, 20 bp, Artificial Sequence
  Description of Artificial Sequence Synthetic primer
acgagatacgtacgcctttg

SEQ ID NO: 29 - DNA, 19 bp, Artificial Sequence
  Description of Artificial Sequence Synthetic primer
tcccggttattttaaggcg

SEQ ID NO: 30 - DNA, 20 bp, Artificial Sequence
  Description of Artificial Sequence Synthetic primer
ccagcaagcatgtcaatgta

SEQ ID NO: 31 - DNA, 20 bp, Artificial Sequence
  Description of Artificial Sequence Synthetic primer
gcatcggtcctaactaaggg

SEQ ID NO: 32 - DNA, 20 bp, Artificial Sequence
  Description of Artificial Sequence Synthetic primer
ggagctttgttcttgcgaac

SEQ ID NO: 33 - DNA, 20 bp, Artificial Sequence
  Description of Artificial Sequence Synthetic primer
tgcaagcagcagatttagtg

SEQ ID NO: 34 - DNA, 20 bp, Artificial Sequence
  Description of Artificial Sequence Synthetic primer
tcgggaaaacctaccctacc

SEQ ID NO: 35 - DNA, 18 bp, Artificial Sequence
  Description of Artificial Sequence Synthetic primer
gcgttggttggacctgac

SEQ ID NO: 36 - DNA, 20 bp, Artificial Sequence
  Description of Artificial Sequence Synthetic primer
atggttggtaggcaccaaag

SEQ ID NO: 37 - DNA, 21 bp, Artificial Sequence
  Description of Artificial Sequence Synthetic primer
cggtgagacagaatccttacg

SEQ ID NO: 38 - DNA, 48 bp, Oryza sativa
aaagagagagagagagagagagagagagagagaggggagggagagagg

SEQ ID NO: 39 - DNA, 48 bp, Oryza sativa; identical to SEQ ID NO: 38

SEQ ID NO: 40 - DNA, 48 bp, Oryza sativa; identical to SEQ ID NO: 38

SEQ ID NO: 41 - DNA, 50 bp, Oryza sativa
aaagagagagagagagagagagagagagagagagaggggagggagagagg

SEQ ID NO: 42 - DNA, 33 bp, Oryza sativa
aaagagagagagagagagagagagggagagagg

SEQ ID NO: 43 - DNA, 33 bp, Oryza sativa; identical to SEQ ID NO: 42

SEQ ID NO: 44 - DNA, 43 bp, Oryza sativa
aaagagtgagagagagagagagagagagagagagagagagagg

SEQ ID NO: 45 - DNA, 43 bp, Oryza sativa; identical to SEQ ID NO: 44

SEQ ID NO: 46 - DNA, 43 bp, Oryza sativa; identical to SEQ ID NO: 44

SEQ ID NO: 47 - DNA, 43 bp, Oryza sativa; identical to SEQ ID NO: 44

SEQ ID NO: 48 - DNA, 43 bp, Oryza sativa; identical to SEQ ID NO: 44

SEQ ID NO: 49 - DNA, 43 bp, Oryza sativa; identical to SEQ ID NO: 44

SEQ ID NO: 50 - DNA, 50 bp, Oryza sativa
atgggatggagagagagagagagagagagagagagagagagagagccgga

SEQ ID NO: 51 - DNA, 50 bp, Oryza sativa; identical to SEQ ID NO: 50

SEQ ID NO: 52 - DNA, 50 bp, Oryza sativa; identical to SEQ ID NO: 50

SEQ ID NO: 53 - DNA, 50 bp, Oryza sativa; identical to SEQ ID NO: 50

SEQ ID NO: 54 - DNA, 50 bp, Oryza sativa; identical to SEQ ID NO: 50

SEQ ID NO: 55 - DNA, 50 bp, Oryza sativa; identical to SEQ ID NO: 50

SEQ ID NO: 56 - DNA, 56 bp, Oryza sativa
atgggatggagagagagagagagagagagagagagagagagagagagagagccgga

SEQ ID NO: 57 - DNA, 56 bp, Oryza sativa; identical to SEQ ID NO: 56

SEQ ID NO: 58 - DNA, 56 bp, Oryza sativa; identical to SEQ ID NO: 56

SEQ ID NO: 59 - DNA, 60 bp, Oryza sativa
atgggatggagagagagagagagagagagagagagagagagagagagagagagagccgga

SEQ ID NO: 60 - DNA, 60 bp, Oryza sativa; identical to SEQ ID NO: 59

SEQ ID NO: 61 - DNA, 60 bp, Oryza sativa; identical to SEQ ID NO: 59

SEQ ID NO: 62 - DNA, 25 bp, Oryza sativa
gagagagagagagagagagagagag

SEQ ID NO: 63 - DNA, 27 bp, Oryza sativa
gagagagagagagagagagagagagag

SEQ ID NO: 64 - DNA, 25 bp, Oryza sativa
gagagagagagagagagagagagag

SEQ ID NO: 65 - DNA, 23 bp, Oryza sativa
gagagagagagagagagagagag

SEQ ID NO: 66 - DNA, 27 bp, Oryza sativa
gagagagagagagagagagagagagag

SEQ ID NO: 67 - DNA, 43 bp, Oryza sativa
gagagagagagagagagagagagagagagagagagagagagag

SEQ ID NO: 68 - DNA, 51 bp, Oryza sativa
gagagagagagagagagagagagagagagagagagagagagagagagagag

SEQ ID NO: 69 - DNA, 27 bp, Oryza sativa
gagagagagagagagagagagagagag

SEQ ID NO: 70 - DNA, 59 bp, Oryza sativa
gagagagagagagagagagagagagagagagagagagagagagagagagagagagagag

SEQ ID NO: 71 - DNA, 45 bp, Oryza sativa
gagagagagagagagagagagagagagagagagagagagagagag

SEQ ID NO: 72 - DNA, 49 bp, Oryza sativa
gagagagagagagagagagagagagagagagagagagagagagagagag

SEQ ID NO: 73 - DNA, 49 bp, Oryza sativa; identical to SEQ ID NO: 72

SEQ ID NO: 74 - DNA, 30 bp, Oryza sativa
acgatggatggatggatggatggatgggtt

SEQ ID NO: 75 - DNA, 30 bp, Oryza sativa; identical to SEQ ID NO: 74

SEQ ID NO: 76 - DNA, 30 bp, Oryza sativa; identical to SEQ ID NO: 74

SEQ ID NO: 77 - DNA, 30 bp, Oryza sativa; identical to SEQ ID NO: 74

SEQ ID NO: 78 - DNA, 30 bp, Oryza sativa; identical to SEQ ID NO: 74

SEQ ID NO: 79 - DNA, 38 bp, Oryza sativa
acgatggatggatggatggatggatggatggatggatt

SEQ ID NO: 80 - DNA, 38 bp, Oryza sativa; identical to SEQ ID NO: 79

SEQ ID NO: 81 - DNA, 38 bp, Oryza sativa; identical to SEQ ID NO: 79

SEQ ID NO: 82 - DNA, 38 bp, Oryza sativa; identical to SEQ ID NO: 79

SEQ ID NO: 83 - DNA, 26 bp, Oryza sativa
acgatggatggatggatggatggatt

SEQ ID NO: 84 - DNA, 26 bp, Oryza sativa
acgatggatggatggatggatggatt

SEQ ID NO: 85 - DNA, 42 bp, Oryza sativa
acgatggatggatggatggatggatggatggatggatggatt

SEQ ID NO: 86 - DNA, 74 bp, Oryza sativa
cgtattattattattatcattattattattattattattattattattattattattattattattattattat

SEQ ID NO: 87 - DNA, 74 bp, Oryza sativa; identical to SEQ ID NO: 86

SEQ ID NO: 88 - DNA, 74 bp, Oryza sativa; identical to SEQ ID NO: 86

SEQ ID NO: 89 - DNA, 74 bp, Oryza sativa; identical to SEQ ID NO: 86

SEQ ID NO: 90 - DNA, 74 bp, Oryza sativa; identical to SEQ ID NO: 86

SEQ ID NO: 91 - DNA, 71 bp, Oryza sativa
cgtattattattattatcattattattattattattattattattattattattattattattattattat

SEQ ID NO: 92 - DNA, 65 bp, Oryza sativa
cgtattattattattatcattattattattattattattattattattattattattattattat

SEQ ID NO: 93 - DNA, 59 bp, Oryza sativa
cgtattattattattatcattattattattattattattattattattattattattat

SEQ ID NO: 94 - DNA, 59 bp, Oryza sativa; identical to SEQ ID NO: 93

SEQ ID NO: 95 - DNA, 59 bp, Oryza sativa; identical to SEQ ID NO: 93

SEQ ID NO: 96 - DNA, 59 bp, Oryza sativa; identical to SEQ ID NO: 93

SEQ ID NO: 97 - DNA, 59 bp, Oryza sativa; identical to SEQ ID NO: 93

SEQ ID NO: 98 - DNA, 61 bp, Oryza sativa
ttaagagagagagagagagagagagagagagagagagagagagagagagagagagagagga

SEQ ID NO: 99 - DNA, 59 bp, Oryza sativa
ttaagagagagagagagagagagagagagagagagagagagagagagagagagagagga

SEQ ID NO: 100 - DNA, 59 bp, Oryza sativa; identical to SEQ ID NO: 99

SEQ ID NO: 101 - DNA, 59 bp, Oryza sativa; identical to SEQ ID NO: 99

SEQ ID NO: 102 - DNA, 59 bp, Oryza sativa; identical to SEQ ID NO: 99

SEQ ID NO: 103 - DNA, 59 bp, Oryza sativa; identical to SEQ ID NO: 99

SEQ ID NO: 104 - DNA, 59 bp, Oryza sativa; identical to SEQ ID NO: 99

SEQ ID NO: 105 - DNA, 59 bp, Oryza sativa; identical to SEQ ID NO: 99

SEQ ID NO: 106 - DNA, 59 bp, Oryza sativa; identical to SEQ ID NO: 99

SEQ ID NO: 107 - DNA, 41 bp, Oryza sativa
ttaagagagagagagagagagagagagagagagagagagga

SEQ ID NO: 108 - DNA, 37 bp, Oryza sativa
ttaagagagagagagagagagagagagagagagagga

SEQ ID NO: 109 - DNA, 37 bp, Oryza sativa; identical to SEQ ID NO: 108

SEQ ID NO: 110 - DNA, 54 bp, Oryza sativa
aagccagaacagcaaacacacacagattacagcagcagcagcagcagcagcgaa

SEQ ID NO: 111 - DNA, 54 bp, Oryza sativa; identical to SEQ ID NO: 110

SEQ ID NO: 112 - DNA, 54 bp, Oryza sativa; identical to SEQ ID NO: 110

SEQ ID NO: 113 - DNA, 54 bp, Oryza sativa; identical to SEQ ID NO: 110

SEQ ID NO: 114 - DNA, 54 bp, Oryza sativa; identical to SEQ ID NO: 110

SEQ ID NO: 115 - DNA, 45 bp, Oryza sativa
aagctagaacagcaaacacacacagattacagcagcagcagcgaa

SEQ ID NO: 116 - DNA, 45 bp, Oryza sativa; identical to SEQ ID NO: 115

SEQ ID NO: 117 - DNA, 45 bp, Oryza sativa; identical to SEQ ID NO: 115

SEQ ID NO: 118 - DNA, 45 bp, Oryza sativa; identical to SEQ ID NO: 115

SEQ ID NO: 119 - DNA, 45 bp, Oryza sativa; identical to SEQ ID NO: 115

SEQ ID NO: 120 - DNA, 45 bp, Oryza sativa; identical to SEQ ID NO: 115

SEQ ID NO: 121 - DNA, 45 bp, Oryza sativa; identical to SEQ ID NO: 115

SEQ ID NO: 122 - DNA, 75 bp, Oryza sativa
aagagagagagagagagagagagagagagagagagagagagagagagagagagagagagagagagagagagagat

SEQ ID NO: 123 - DNA, 71 bp, Oryza sativa
aagagagagagagagagagagagagagagagagagagagagagagagagagagagagagagagagagagat

SEQ ID NO: 124 - DNA, 71 bp, Oryza sativa; identical to SEQ ID NO: 123

SEQ ID NO: 125 - DNA, 63 bp, Oryza sativa
aagagagagagagagagagagagagagagagagagagagagagagagagagagagagagagat

SEQ ID NO: 126 - DNA, 59 bp, Oryza sativa
aagagagagagagagagagagagagagagagagagagagagagagagagagagagagat

SEQ ID NO: 127 - DNA, 59 bp, Oryza sativa; identical to SEQ ID NO: 126

SEQ ID NO: 128 - DNA, 59 bp, Oryza sativa; identical to SEQ ID NO: 126

SEQ ID NO: 129 - DNA, 59 bp, Oryza sativa; identical to SEQ ID NO: 126

SEQ ID NO: 130 - DNA, 59 bp, Oryza sativa; identical to SEQ ID NO: 126

SEQ ID NO: 131 - DNA, 59 bp, Oryza sativa; identical to SEQ ID NO: 126

SEQ ID NO: 132 - DNA, 59 bp, Oryza sativa; identical to SEQ ID NO: 126

SEQ ID NO: 133 - DNA, 57 bp, Oryza sativa
aagagagagagagagagagagagagagagagagagagagagagagagagagagagat

(19) United States
(12) Patent Application Publication
(10) Pub. No.: US 2014/0221224 A1
(43) Pub. Date: Aug. 7, 2014

(54) Single tube multiplex assay for detection and quantification of adulterants in basmati rice samples

(71) Applicant: CENTRE FOR DNA FINGERPRINTING AND DIAGNOSTICS (CDFD), Nampally (IN)

(72) Inventors: Sunil Archak, Nampally (IN); Javaregowda Nagaraju, Nampally (IN)

(73) Assignee: CENTRE FOR DNA FINGERPRINTING AND DIAGNOSTICS (CDFD), Nampally (IN)

(21) Appl. No.: 13/998,516

(22) Filed: Nov. 7, 2013

Related U.S. Application Data
(63) Continuation of application No. 13/530,904, filed on Jun. 22, 2012, now abandoned.
Application No. 13/530,904 is a continuation of application No. 12/842,746, filed on Jul. 23, 2010, now abandoned.
Application No. 12/842,746 is a division of application No. 11/406,257, filed on Apr. 19, 2006, now abandoned.
Application No. 11/406,257 is a continuation-in-part of application No. 10/357,488, filed on Feb. 4, 2003, now abandoned.

(30) Foreign Application Priority Data
Apr. 8, 2002 (IN) 260/MAS/2002

(51) Int. Cl. C12Q 1/68

(52) U.S. Cl. CPC C12Q 1/6895
USPC 506/9

(57) ABSTRACT

The present invention provides a single tube multiplex assay for distinguishing basmati from non-basmati rice varieties, and thereby identifying the adulteration of basmati rice varieties. The present invention further provides a method for quantifying adulteration in basmati rice varieties. The present invention also provides a kit for performing a muliplex assay for distinguishing basmati from non-basmati rice varieties. The kit may comprise a primer directed to an SSR loci, appropriate reagents for PCR, and optionally, a package insert for conducting the assay.

This application is a Rule 53(b) continuation of co-pending U.S. patent application Ser. No. 13/530,904 filed Jun. 22, 2012, which is a continuation of co-pending U.S. application Ser. No. 12/842,746 filed Jul. 23, 2010, which is a divisional of co-pending U.S. patent application Ser. No. 11/406,257 filed Apr. 19, 2006, which is a Continuation-in-Part application of U.S. patent application Ser. No. 10/357,488 filed Feb. 4, 2003, which in turn claims priority to Indian Patent Application No. 260/MAS/2002 filed Apr. 8, 2000. The specifications of all priority applications are incorporated herein by reference.

### FIELD OF THE PRESENT INVENTION

The present invention relates to the assays for detection and quantification of adulterants in basmati rice varieties.

### BACKGROUND AND PRIOR ARTS OF THE PRESENT INVENTION

Traditional basmati varieties command a considerable price advantage in the international market over others. For instance, in European market, Indian traditional Basmati like Dehradun Basmati commands $850 per tonne where as evolved basmati cultivars like Pusa Basmati and Super Basmati get $480 and $500 per tonne respectively, and non-basmati long-grain rice fetch a meagre $160 per tonne. Additionally, some overseas markets encourage varieties that are more authentic by granting duty exemption. For example, in European market, a tariff of $78 per tonne is imposed on husked rice; whereas for nine Basmati varieties, the import duty is completely exempted (European Commission regulation 1549/2004).

Considering the price differences in the light of the total volume of international basmati rice trade (˜1.5 million MT), it is obvious that unscrupulous practices such as adulteration of traditional basmati offer cost advantage to the traders. Since it is not quite easy to differentiate between traditional basmati and other long grain rice varieties, and a label of traditional basmati brings along duty advantage, fraudulent traders make a substantial profit by adulterating traditional basmati with either evolved basmati or non-basmati varieties and exploit the gullible consumer. Such practices have been shown to be existing and rampant by a food survey conducted by the Food Standards Agency of the United Kingdom (world wide web food.gov.uk/science/surveillance/fsis2004branch/fsis4704basmati). The adulteration of traditional basmati grains affects the exporting countries too in terms of the tarnished image and diminished interest in the brands. Hence, to protect the interests of consumers and trade, identification of genuine basmati rice samples and devaluation of adulterated samples becomes vital.

Differentiation of traditional basmati varieties from other long grain varieties based on aroma, chemical composition and grain elongation arc impracticable for large-scale applications. Microsatellite profiles can be used for cultivar identification and detection of adulteration. We have already designated microsatellite profiles of traditional basmati, evolved basmati and non-basmati rice varieties (Nagaraju et al 2002). In fact, importers like European Union have now stipulated that all Basmati imports carry a certificate of purity based on a DNA test.

### OBJECTS OF THE PRESENT INVENTION

The main object of the present invention relates to development of a single tube multiplex assay for distinguishing basmati from non-basmati rice varieties and thereby the adulteration.

Yet another object of the present invention is to develop a method of quantifying adulteration in basmati rice varieties.

### SUMMARY OF THE PRESENT INVENTION

The present invention relates to a single tube multiplex assay for distinguishing basmati from non-basmati rice varieties and thereby the adulteration, said assay comprising steps of running multiplex PCR with sample using one or more loci of Table 3, and distinguishing the basmati from non-basmati rice varieties and thereby the adulteration on the basis of varietal specific multiplex allele profile; and also, a method of quantifying adulteration in basmati rice varieties, said method comprising steps of constructing a standard curve on the basis of ratio of quantity of amplified products of the alleles of adulterant and the basmati rice against the progressive proportion of adulteration, and quantifying the adulteration in basmati rice variety on the basis of peak area of the alleles corresponding to basmati and that of the adulterant.

### DETAILED DESCRIPTION OF THE PRESENT INVENTION

Accordingly, the present invention relates to a single tube multiplex assay for distinguishing basmati from non-basmati rice varieties and thereby the adulteration, said assay comprising steps of running multiplex PCR with sample using one or more loci of Table 3, and distinguishing the basmati from non-basmati rice varieties and thereby the adulteration on the basis of varietal specific multiplex allele profile; and also, a method of quantifying adulteration in basmati rice varieties, said method comprising steps of constructing a standard curve on the basis of ratio of quantity of amplified products of the alleles of adulterant and the basmati rice against the progressive proportion of adulteration, and quantifying the adulteration in basmati rice variety on the basis of peak area of the alleles corresponding to basmati and that of the adulterant.

A set of ten SSR loci has been identified and the competence its allele profiles to genotype various basmati varieties has been demonstrated. Further, a multiplex system to make use of allele size information for the identification of adulterants in commercial samples of basmati rice has been designed. It was also demonstrated that the multiplex system could be used to quantify the adulterant. Here, a high throughput “single tube assay” method based on multiplexing all or a combination of the ten microsatellite markers is described as a tool to certify genuineness of Basmati rice samples as shown in FIG. 7.

1. Identification of the Adulterant

Primary step in the identification of an adulterant is to make unequivocal identification possible by generating variety-specific microsatellite profiles of the basmati varieties designated for trade and possible adulterants (Table 1). 350 primers were screened on the varieties (sequence source: world wide web.gramene.org). Sixteen primers were selected based on amplification of a single and clear band and discrimination power (Table 2). A panel of ten informative microsatellite loci was developed that differentiate various traditional basmati, evolved basmati varieties and others as well as amenable for multiplexing (Table 3). Upon PCR, a genuine sample of a traditional basmati variety yields a single allele of the size listed in the panel. However, any admixture of traditional basmati with either evolved basmati or non-basmati would be detected at least at one of the microsatellite loci because of different allele sizes. Subsequently, we arranged these primers based on allele sizes in such a way that using 3 fluorescent ligands in the PCR primers we could run a single genotyping assay. The above two steps resulted in a methodology where, (a) Pure samples of all varieties could be unequivocally identified, and (b) Allele pattern could also identify the varietal mixtures.

2. Construction of Standard Curve and Quantitation of Adulterant

It is possible that some basmati rice samples may contain adventitious mixture as a result of inadvertent mixing in the field/storage. If we can measure the actual amount of the adulterant, such samples having admixture within limits allowed by the importing countries (for instance, 7% recommended by The Grain and Feed Trade Association, GAFTA Code of Practice for Rice) could be certified as practically genuine. Therefore, we went a step ahead in our effort and designed experiments to actually quantify the adulterant in basmati rice samples.

Given the differentiating alleles between the traditional basmati (major component) and evolved basmati or non-basmati (adulterant), the quantitation procedure was based on the premise that if we can quantify the amplified allelic products of a “common locus”, the ratio between quantities of the amplicons can reveal the ratio of the quantities of competing DNA templates in a PCR mixture. The procedure involved preparation of a series of standards of traditional basmati rice samples with a progressive proportion of adulteration. The approach was to generate a “standard curve” by plotting the ratio of the quantity of amplified products of the alleles of adulterant and the traditional Basmati against the progressive proportion of adulteration. Quantity of the amplified allele was calculated based on the peak area of the allele obtained on the electropherogram.

### BRIEF DESCRIPTION OF THE ACCOMPANYING DRAWINGS

The patent or application file contains at least one drawing executed in color. Copies of this patent or patent application publication with color drawing(s) will be provided by the Office upon request and payment of the necessary fee.

FIG. 1 shows allelic profile of Basmati 370 obtained by single assay multiplex reaction. Three colours represent three groups of primers labelled with specific fluorescent ligands (blue is FAM, Green is JOE and black is TAMRA). Locus name and allelic size in base pairs are given below the peaks; FIG. 1A shows allelic profiles of pure basmati 370, pure adulterant sharbati and an adulterant sample, by using only two primers from the multiplex panel.

FIG. 2 shows sequence alignment of alleles of RM55 locus from different rice varieties (viz. basmati, evolved basmati and non-basmati long grain rice varieties) showing variation in length. The sequences in FIG. 2 from top to bottom are represented as SEQ ID NOS: 38-49.

FIG. 3 shows standard curve generated for a combination of basmati 370 adulterated with sharbati using allele differences at RM 348 locus.

FIG. 4 shows mixing experiments (Ranbir: Pusa basmati) in different combinations and the peaks obtained thereby at particular loci (RM348, RM171 and RM72, respectively).

FIGS. 5 and 5a-5g show photographs of Agarose Gel run to establish amplification for various PCRs. In particular, FIGS. 5 and 5a-5g depict Agarose Gel run results of PCR products of different rice varieties (viz. basmati, evolved basmati and non-basmati long grain rice varieties) amplified at various loci [viz. RM55 (FIG. 5), RM44 (FIG. 5a), RM1 (FIG. 5b), RM171 (FIG. 5c), RM72 (FIG. 5d), RM202 (FIG. 5e), RM348 (FIG. 5f) and RM241 (FIG. 5g).

FIGS. 6a-6h show sequence alignment data of various loci from different rice varieties (viz. basmati, evolved basmati and non-basmati long grain rice varieties).

FIG. 6a shows sequence alignment of alleles of RM55 locus. The sequences of FIG. 6a from top to bottom are represented as SEQ ID NOS: 38-94.

FIG. 6b shows sequence alignment of alleles of RM44 locus. The sequences of FIG. 6b from top to bottom are represented as SEQ ID NOS: 50-61.

FIG. 6c shows sequence alignment of alleles of RM1 locus. The sequences of FIG. 6c from top to bottom are represented as SEQ ID NOS: 62-73.

FIG. 6d shows sequence alignment of alleles of RM171 locus. The sequences of FIG. 6d from top to bottom are represented as SEQ ID NOS: 74-85.

FIG. 6e shows sequence alignment of alleles of RM72 locus. The sequences of FIG. 6e from top to bottom are represented as SEQ ID NOS: 86-97.

FIG. 6f shows sequence alignment of alleles of RM202 locus. The sequences of FIG. 6f from top to bottom are represented as SEQ ID NOS: 98-109.

FIG. 6g shows sequence alignment of alleles of RM348 locus. The sequences of FIG. 6g from top to bottom are represented as SEQ ID NOS: 110-121.

FIG. 6h shows sequence alignment of alleles of RM241 locus. The sequences of FIG. 6h from top to bottom are represented as SEQ ID NOS: 122-133.

FIGS. 7, 7a-7f, 8 and 8a-8c show multiplex mixing combination results. In particular, allelic profiles of different rice varieties (viz. basmati, evolved basmati and non-basmati long grain rice varieties) obtained by single assay multiplex reaction at 8 loci (RM1, RM72, RM171, RM241, RM202, RM44, RM348 and RM55) is depicted. FIG. 7 relates to the allelic profile of Basmati370; FIG. 7a relates to the allelic profile of Dehradun basmati; FIG. 7b relates to the allelic profile of Taraori basmati; FIG. 7c relates to the allelic profile of Basmati386; FIG. 7d relates to the allelic profile of Ranbir basmati;

FIG. 7e relates to the allelic profile of Basmati217; FIG. 7f relates to the allelic profile of Haryana basmati; FIG. 8 relates to the allelic profile of Pusa basmati; FIG. 8a relates to the allelic profile of Sharbati; FIG. 8b relates to the allelic profile of Jaya; and FIG. 8c relates to the allelic profile of IR64.

### BRIEF DESCRIPTION OF THE TABLES OF THE PRESENT INVENTION

Table 1 shows list of varieties used for standardisation of multiplex.

Table 2 shows SSR loci (including those added in the CIP) that are selected to distinguish basmati from non-basmati subsequent to large-scale screening.

Table 3 shows the panel of ten informative SSR loci selected for multiplex assay.

Table 4 shows allele sizes (in base pairs) of various basmati rice varieties obtained by multiplex single assay method.

Table 5 shows Genotype codes of various basmati rice varieties based on single assay multiplex method. The order of codes from left to right correspond to loci 1 to 8 given in Table 5.

Table 6 shows Allele sizes in base pairs for corresponding codes of Table 4.

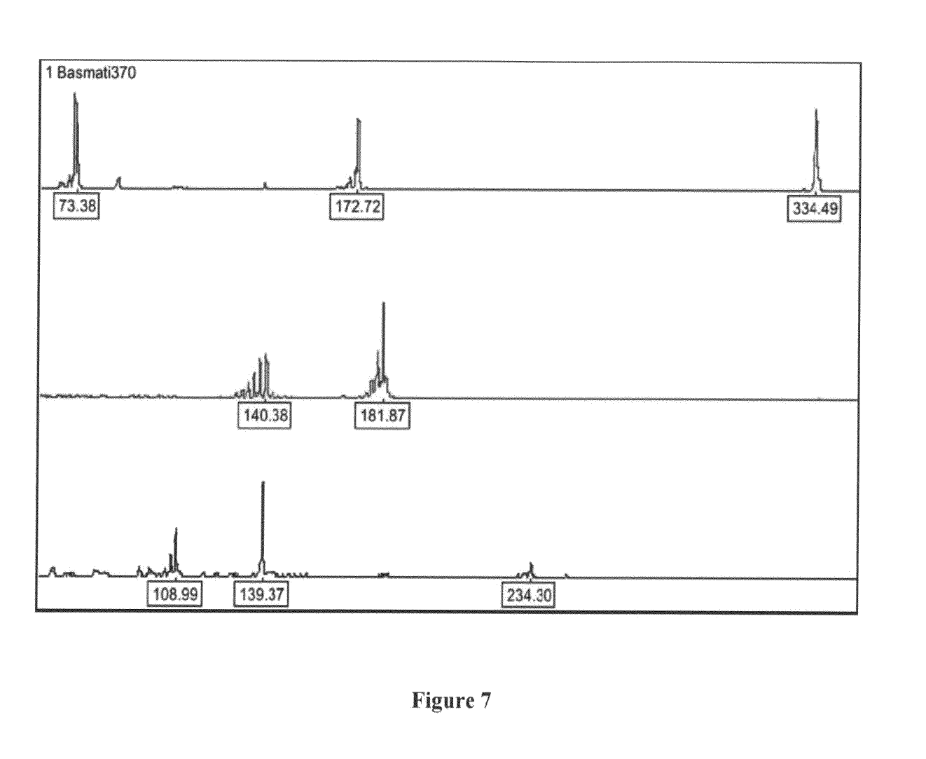

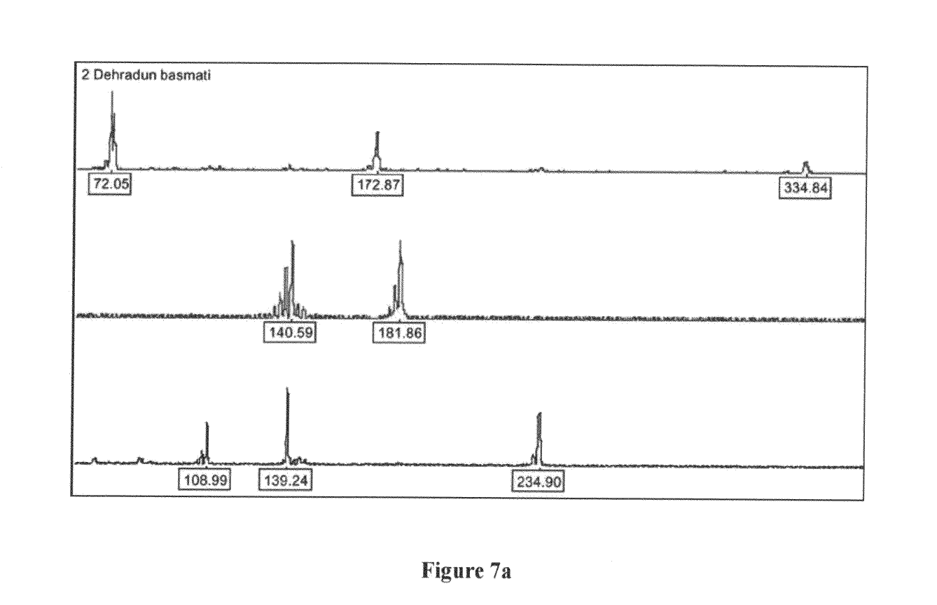

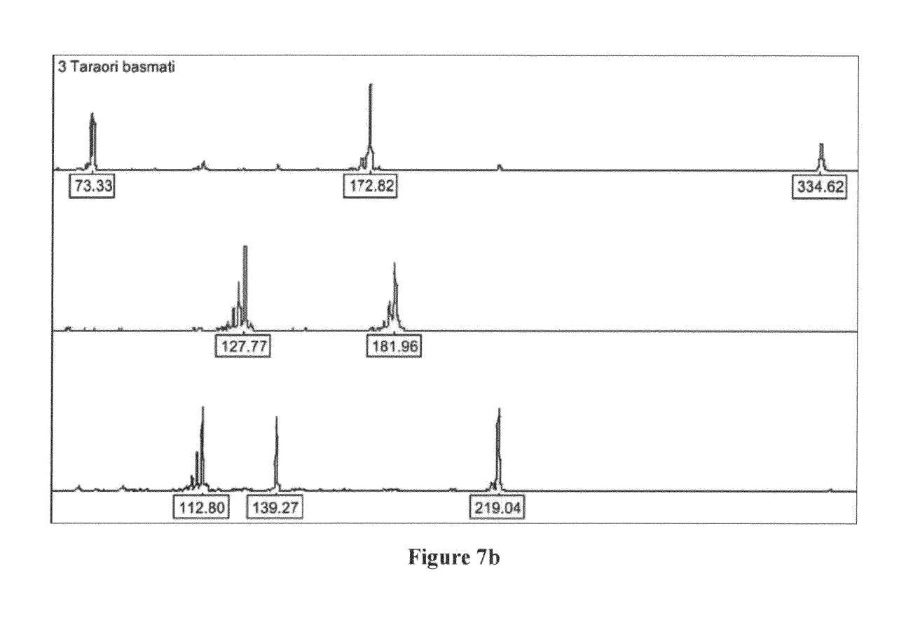

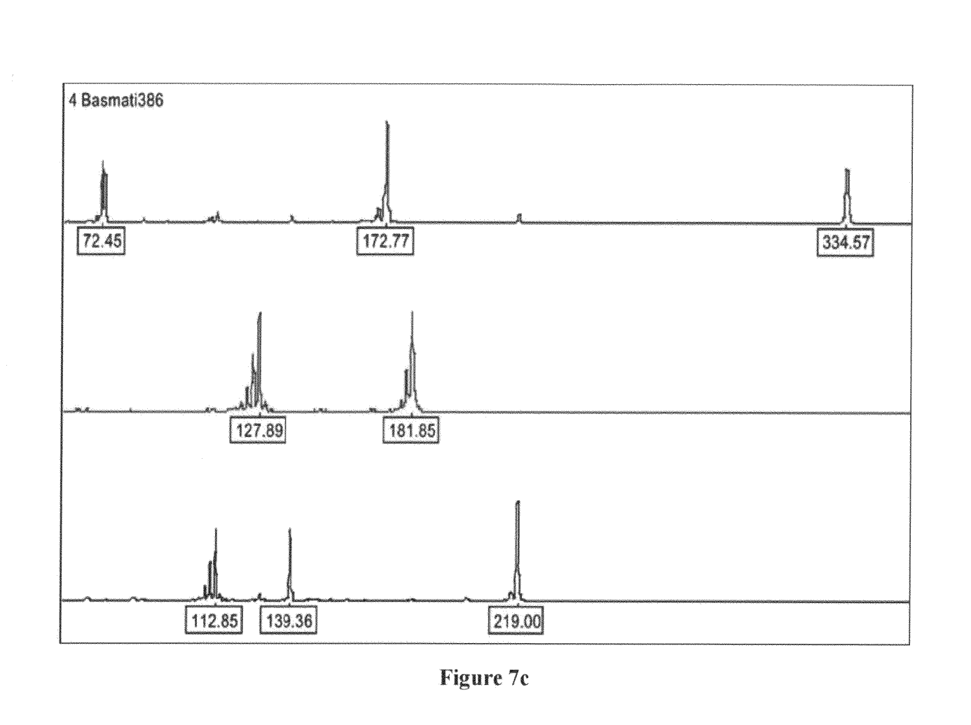

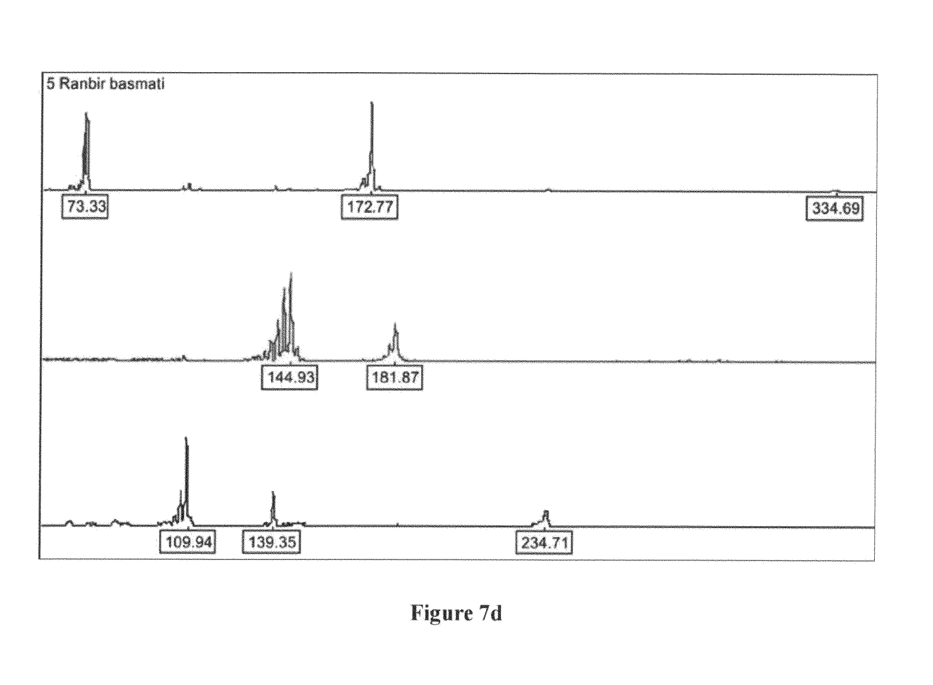

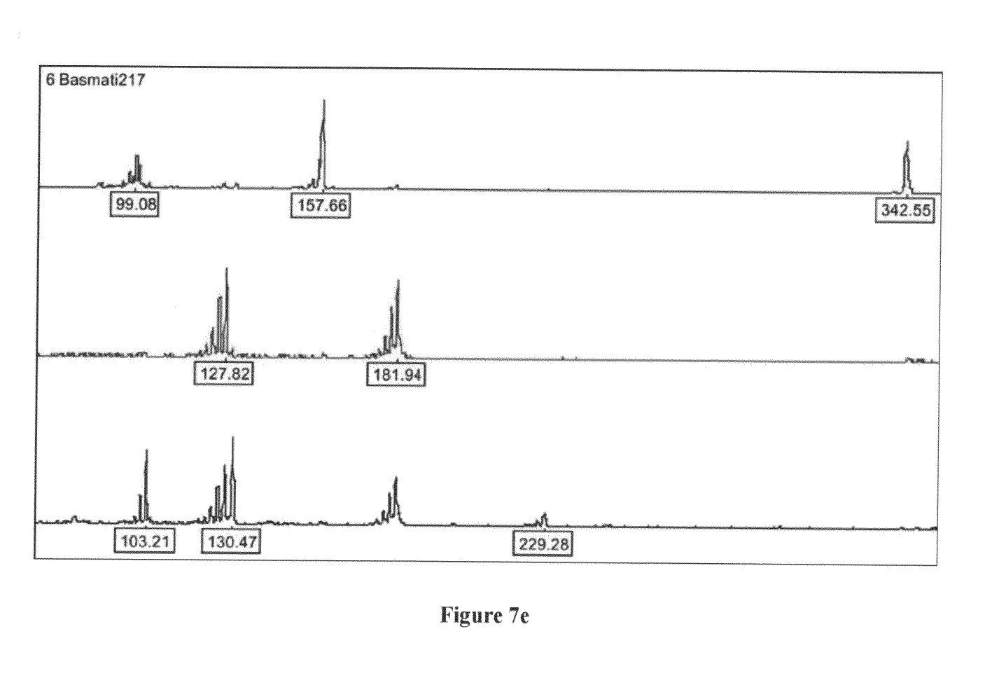

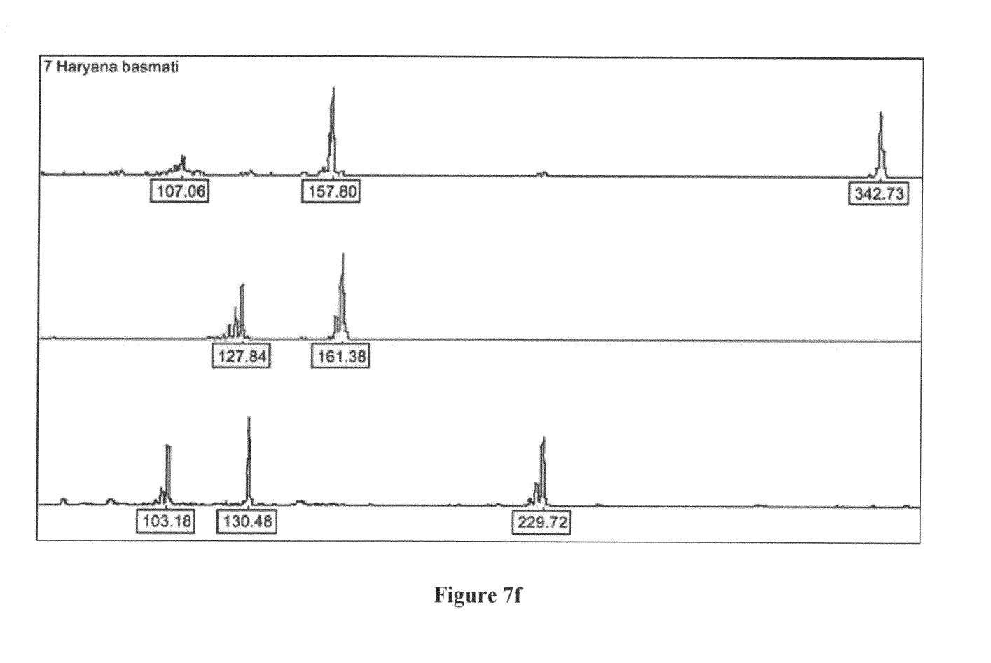

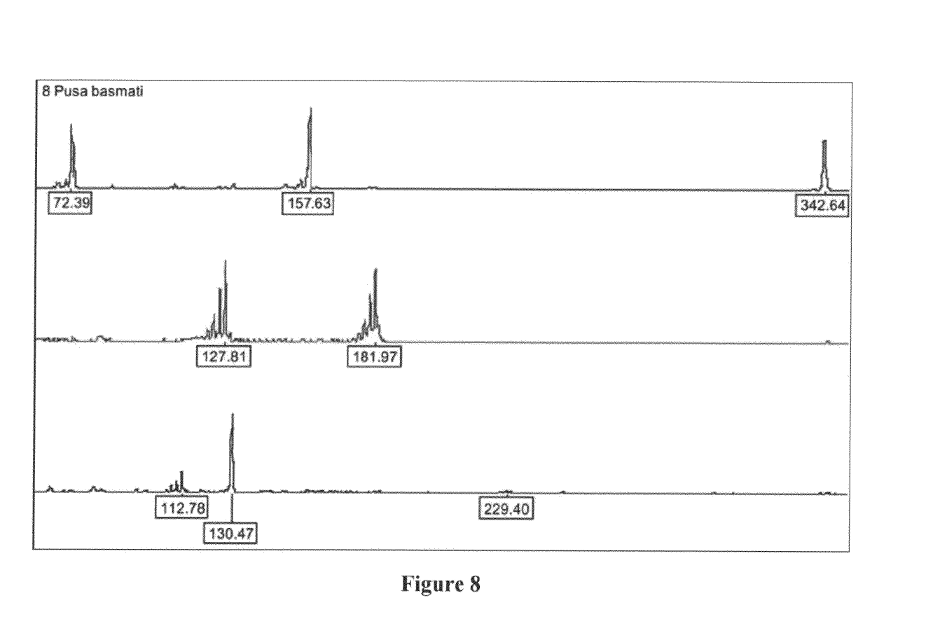

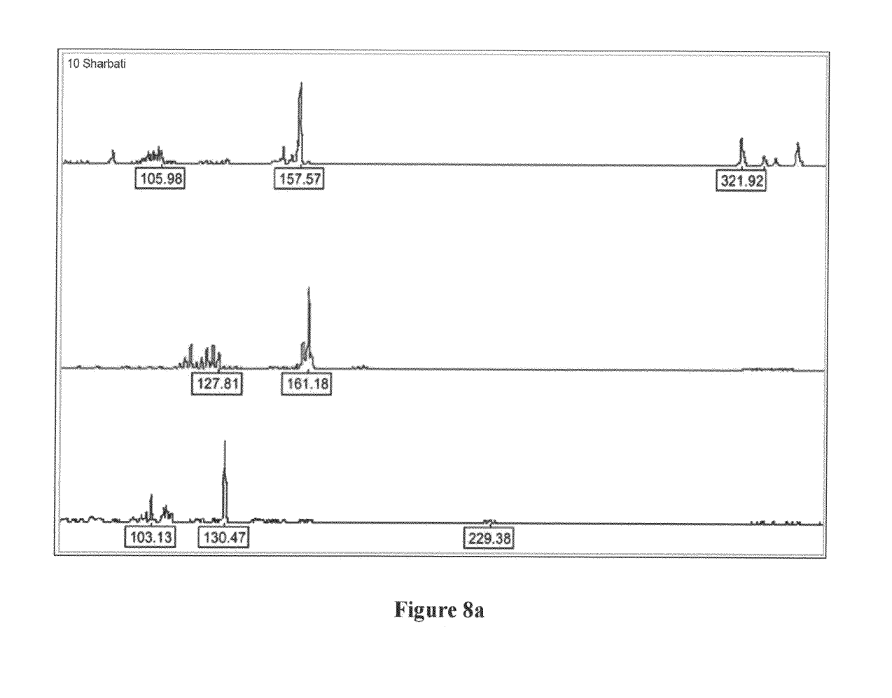

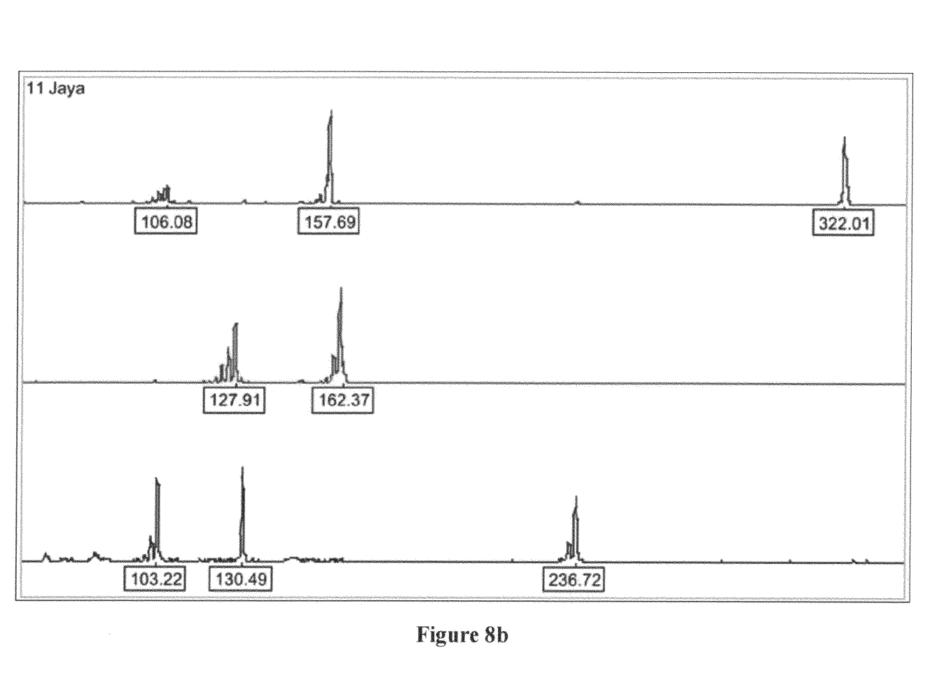

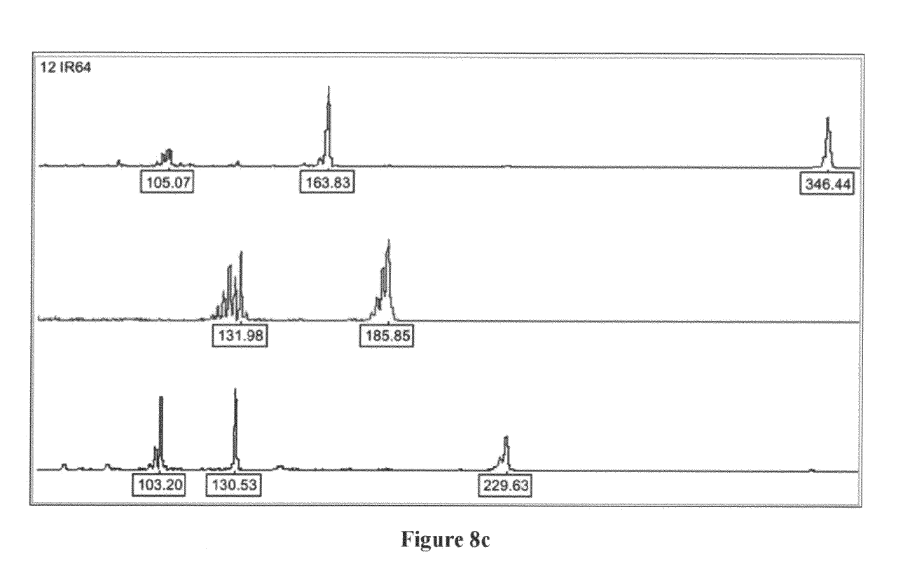

Table 7 Shows how these 10 primers were arranged in a particular manner to facilitate single genotyping assay. It is clear from the table that loci were grouped so as to avoid overlapping allele sizes in the same fluorescence label (read as ‘same coloured peaks in the electrophoresis’) as shown in FIG. 7.

The loci could be employed to distinguish basmati and non-basmati in a ‘single tube assay’ is the result of the present research. The number of markers would vary from case to case and thus, the requirement can vary from 1 to all the 10 markers. The assay can differentiate any two known varieties using only one locus. However, a combination of the markers is employed in a multiplex single tube reaction to identify the main variety and any combination of adulterants in the genuine basmati grains.

The web link for the rice microsatellite primer list is world wide web.gramene.org/microsat/ssr.html. This site had only 350 loci when the study was initiated, but now contains nearly two thousand microsatellite loci.

Experimental Data on the Basis of which 10 Markers were Selected is Provided Below.

1. Preliminary screening of the loci was done for the amplification of a clear and single amplicon. Those loci, at which a) no amplification b) non-specific amplification c) stutter problem and, d) inconsistent amplification were obtained were eliminated.

2. In the second step of screening only those loci for which primer pairs have annealing temperature of at least 55° C. were selected to ensure stringent PCR conditions in the assay.

3. Ideally such loci were selected that generated more than two alleles and could be easily differentiated from stutters if any.

4. Loci generating private alleles specific to particular variety were given preference.

5. Among the most distinguishing loci, those with high reproducibility of the allele size were selected for further analysis.

6. The loci were then tested for existence of polymorphism among and between basmati genotypes especially a set of the varieties that are commercially important.

Comprehensive details of the experimental data to arrive at the “Standard Curve” are provided as given below. In addition, shown are standard curve experiments for other combinations also, apart from Basmati 370 and Sharbati using locus RM348. Here, calculations are also provided to arrive at Peak Area and also, the percentage adulteration determined in such cases.

Construction of Standard Curve and Quantitation of the Adulterant

It is possible that some basmati rice samples contain adventitious mixture because of inadvertent mixing in the field/storage. If we can measure the actual amount of the adulterant, such samples having admixture within limits allowed by the importing countries (for instance, 7% recommended by The Grain and Feed Trade Association, GAFTA Code of Practice for Rice) could be certified as practically genuine. Therefore, we designed experiments to actually quantify the adulterant in basmati rice samples.

Given the differentiating alleles between the traditional basmati (major component) and evolved basmati or non-basmati (adulterant), the quantitation procedure is based on the premise that if we can quantify the amplified allelic products of a “common locus”, the ratio between quantities of the amplicons can reveal the ratio of the quantities of competing DNA templates in the PCR mixture. The procedure involved preparation of a series of standards of traditional basmati rice samples with a progressive proportion of adulteration. The approach was to generate a “standard curve” by plotting the ratio of the quantities of amplified products of adulterant and the traditional Basmati alleles against the degree of adulteration. Quantity of the amplified allele was calculated based on the peak area of the allele obtained on the electropherogram.

Standard curves were constructed for a combination of Basmati370:Sharbati mixtures at two discriminating loci, RM72 and RM348. Standard samples were prepared by mixing the grains of the Basmati370 with Sharbati at progressive ratio of 1%, 3%, 5%, 7%, 10%, 15%, 17%, 20%, 25%, 30%, 40% and 60% to generate data at 12 score points. Subsequent to genotyping, peak areas were determined for each score point and were plotted against the percent adulterant to develop a standard curve based on logistic model (y=a/l+be^{-cx}) by using CurveExpert 1.38 (http://curveexpert.webhop.net). A standard curve was also generated by mixing DNA isolated from the milled grains of Sharbati, a common adulterant, in various ratio at 5%, 10%, 20%, 30%, 40%, 50% and 60% to Basmati370 DNA to generate seven score points on the curve. Systematic bias associated with the employment of standard curves was calculated. The differences were averaged over three independent runs to compute the bias (b) at each score point. Bias (B) introduced by using standard curve was computed as, B=√Σb².

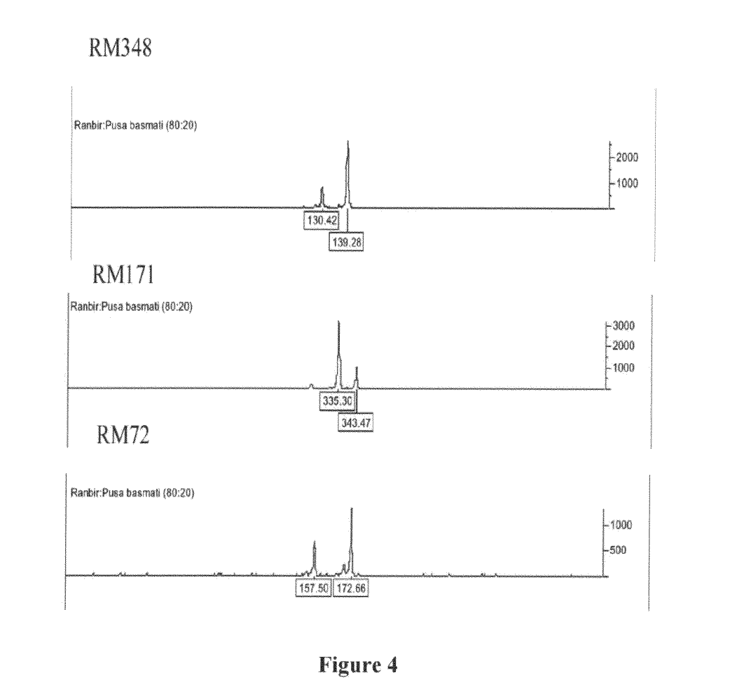

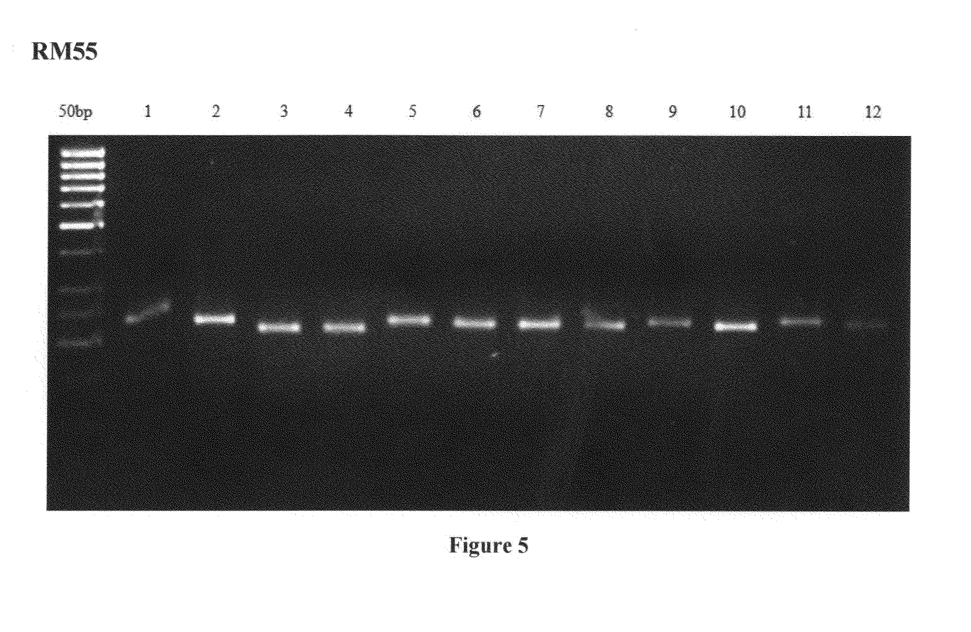

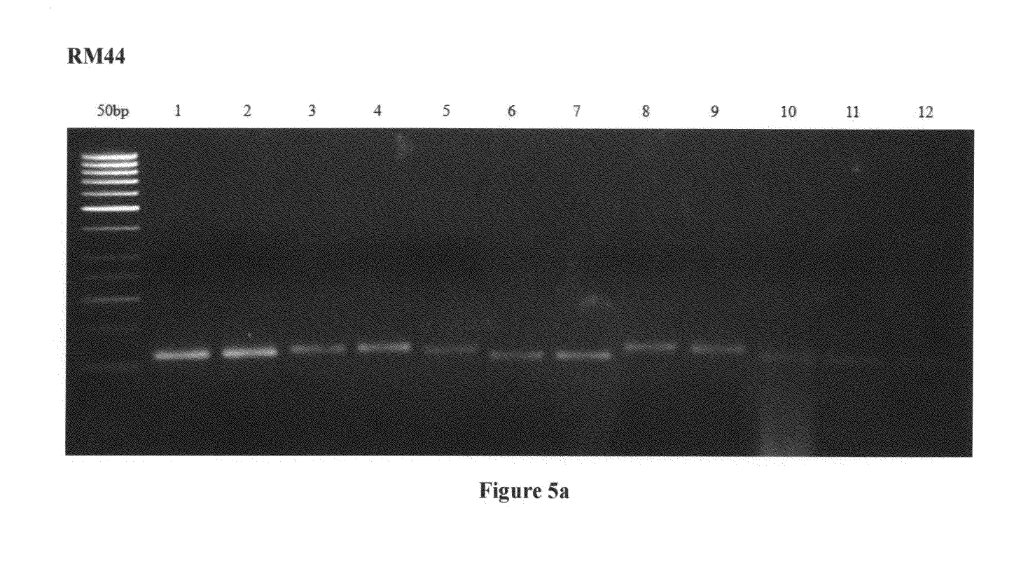

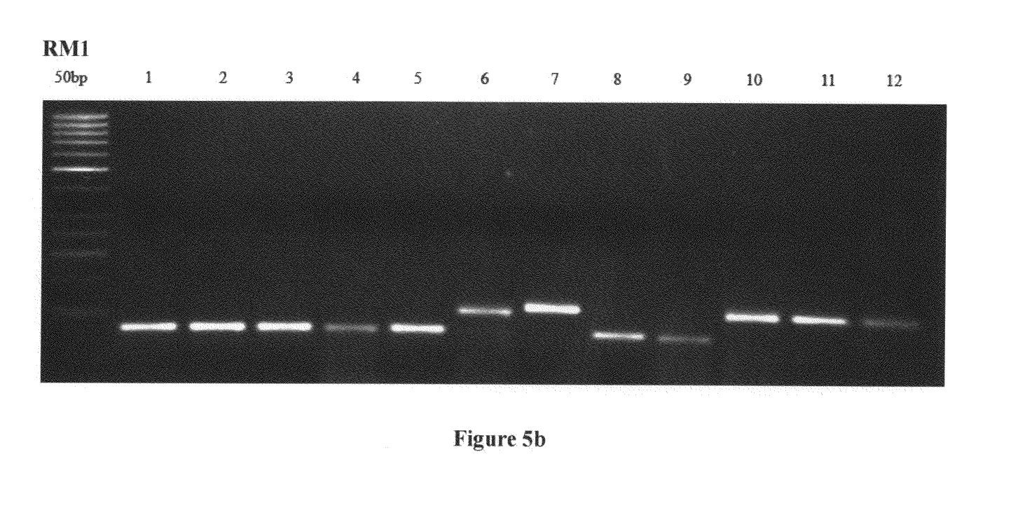

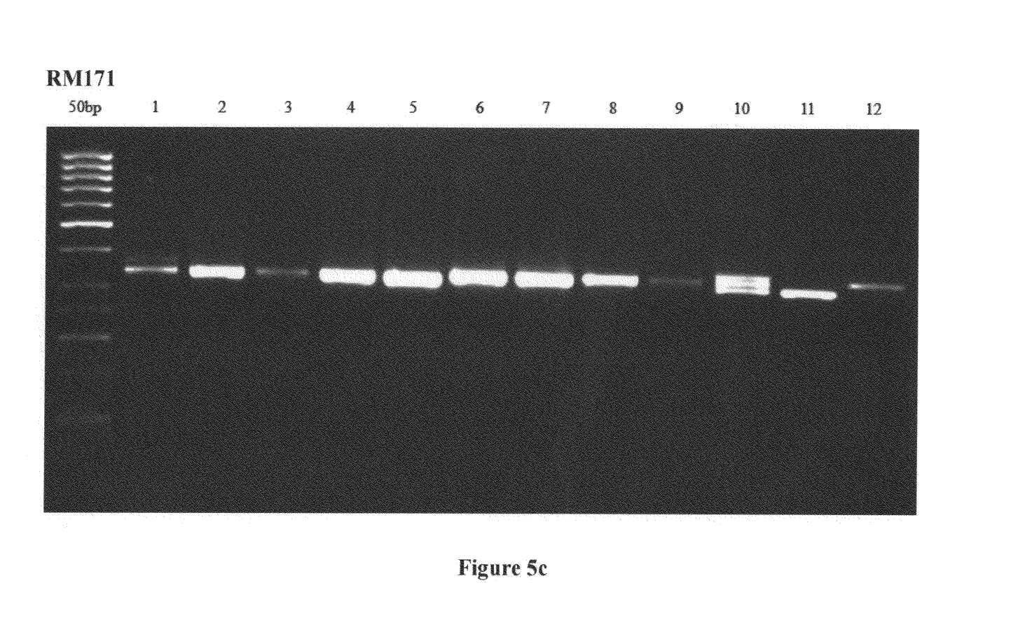

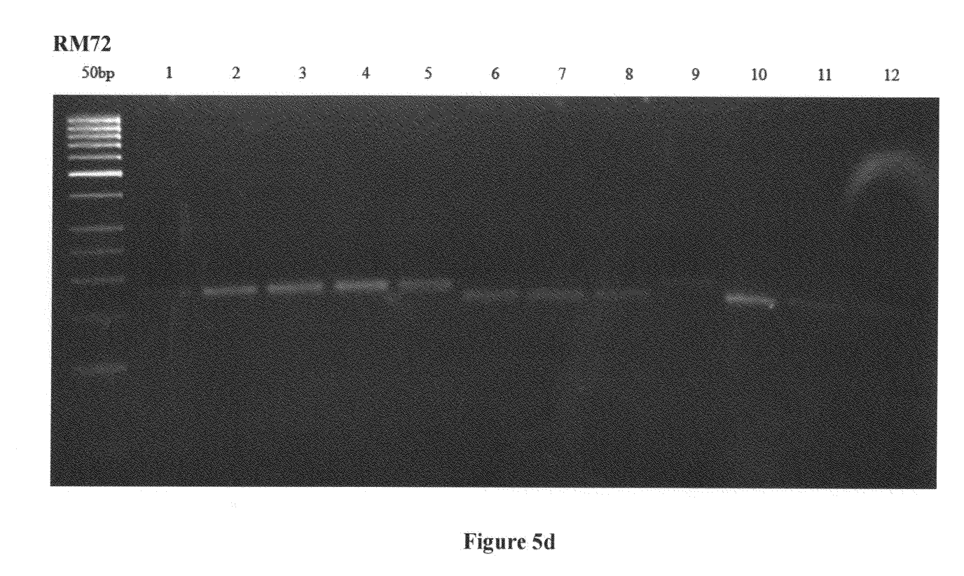

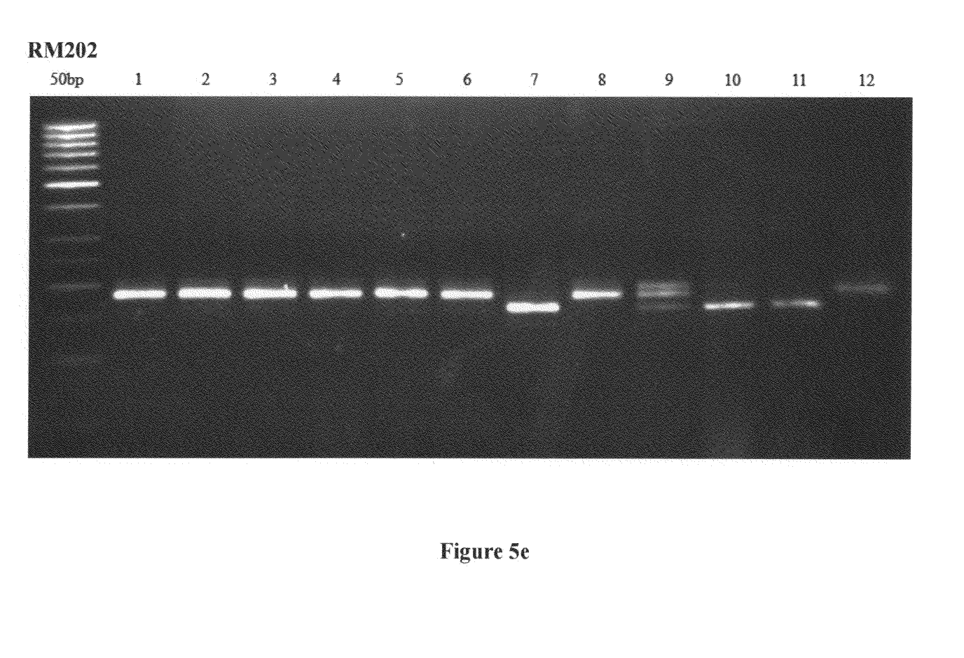

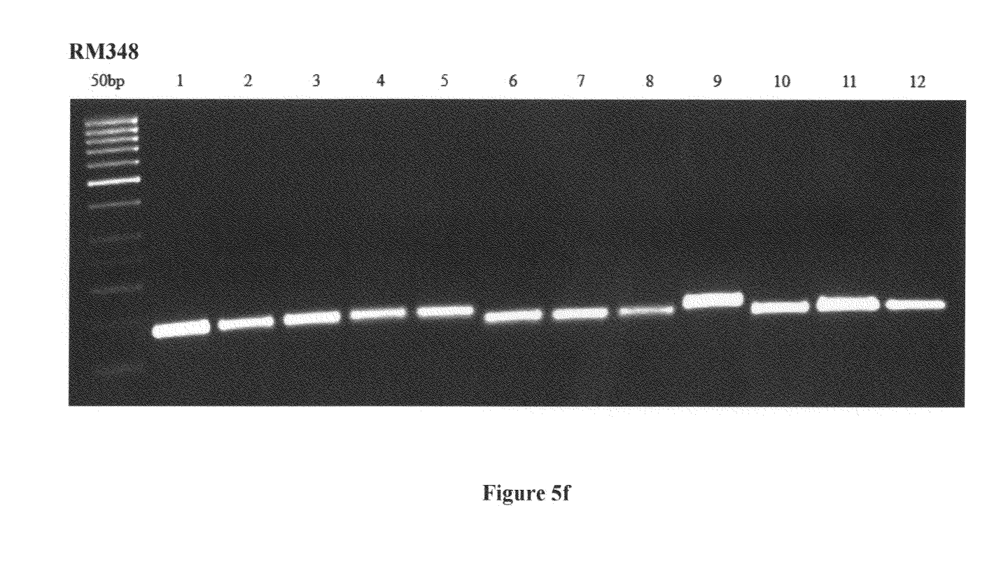

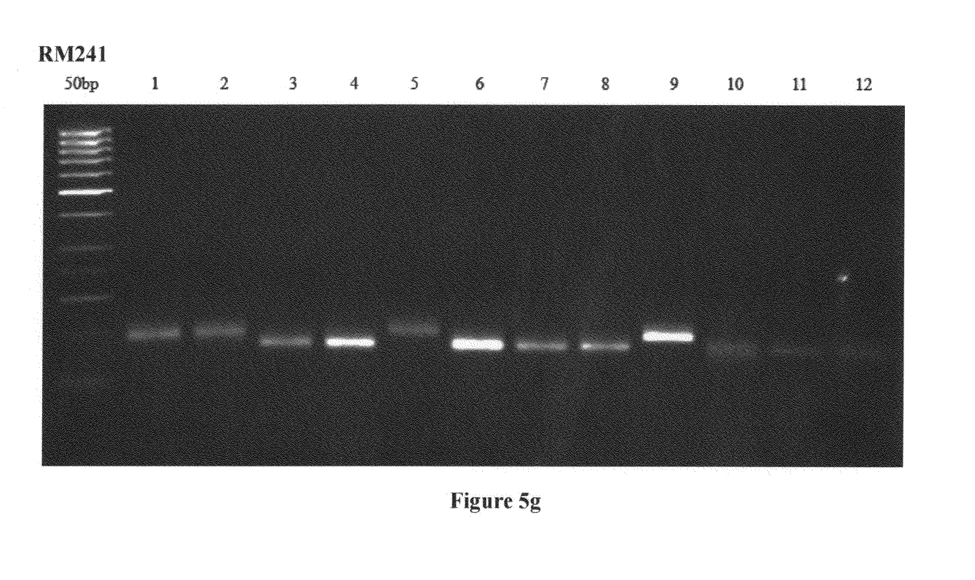

For illustrating mixing experiments in different combinations, peaks obtained at particular combinations are given as FIG. 4. Further, photographs of Agarose Gel run to establish amplification for various PCRs is provided as FIG. 5.

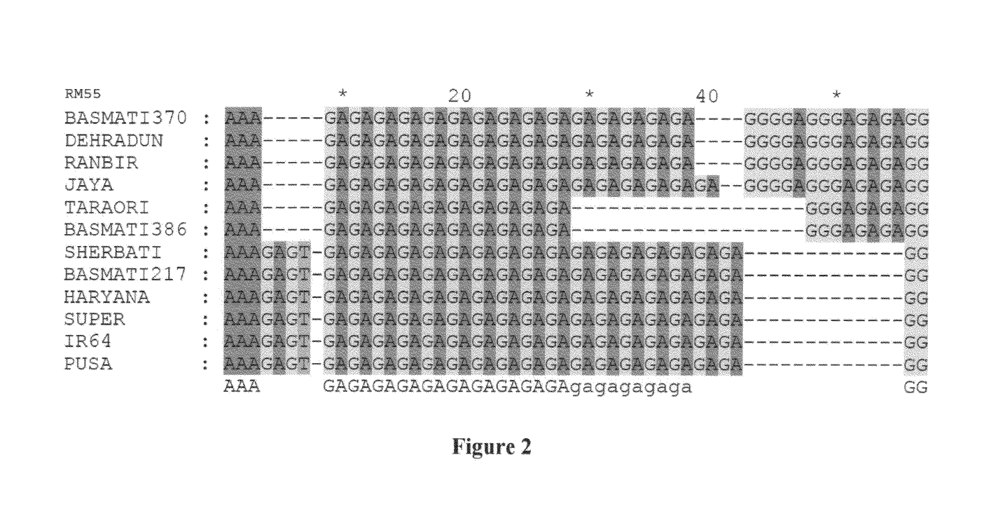

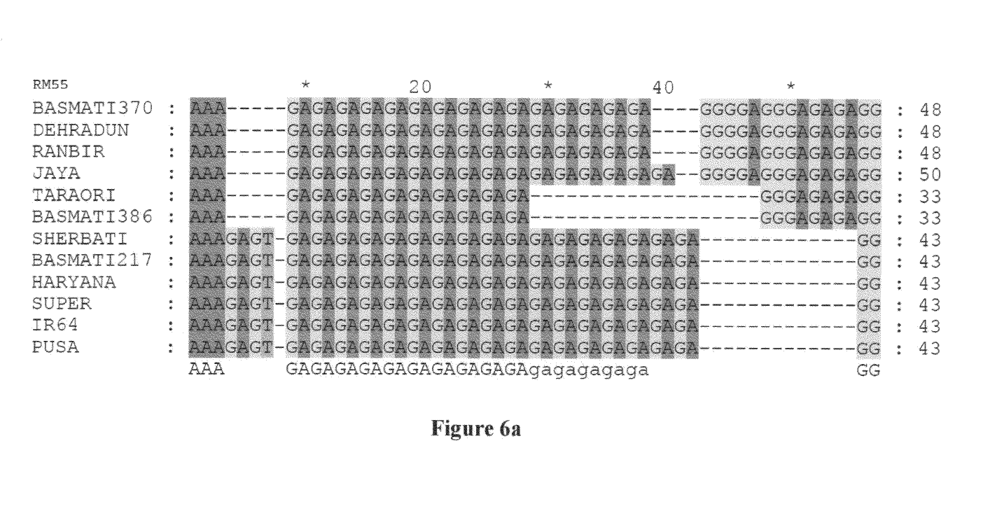

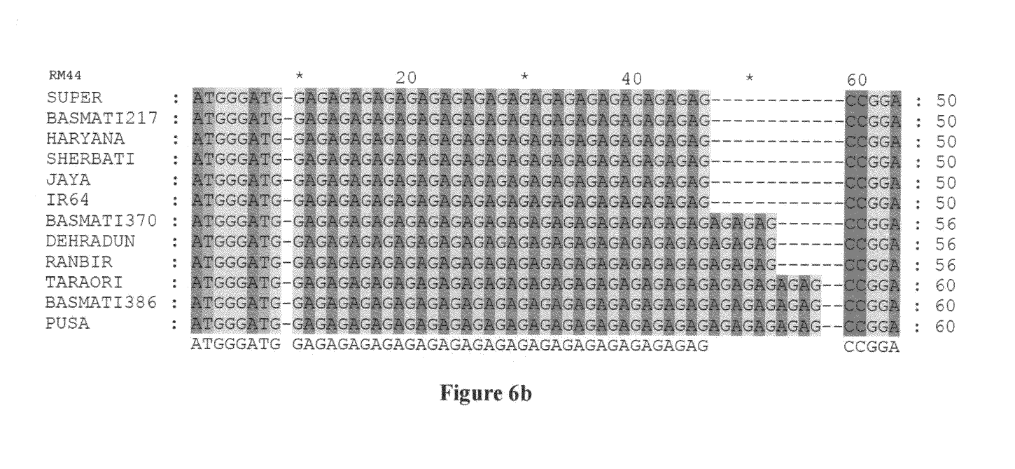

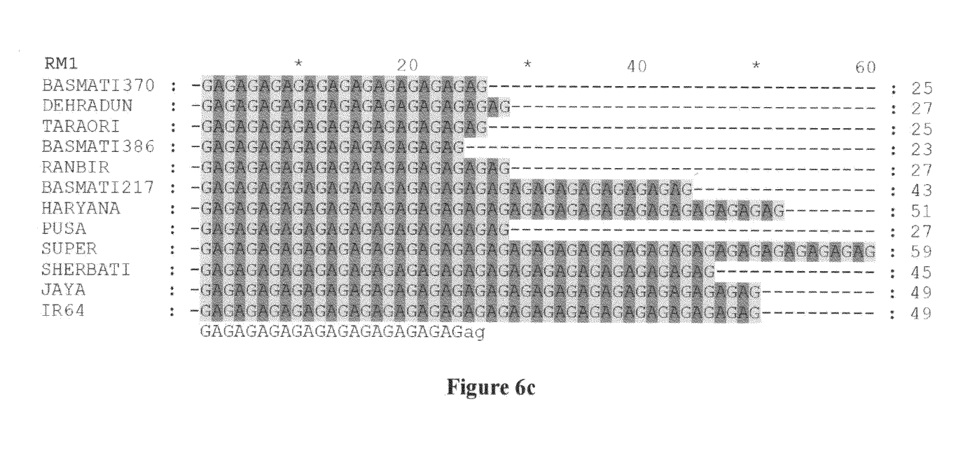

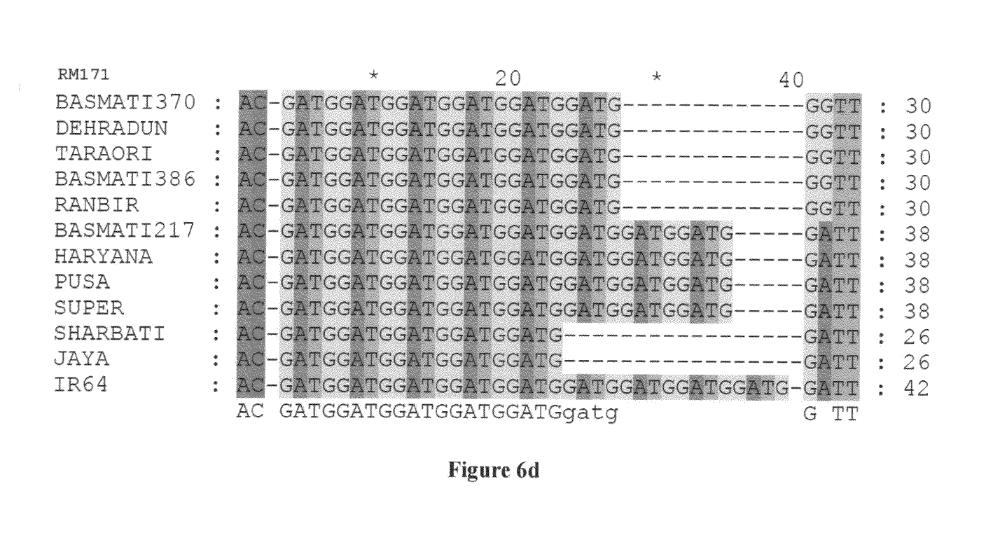

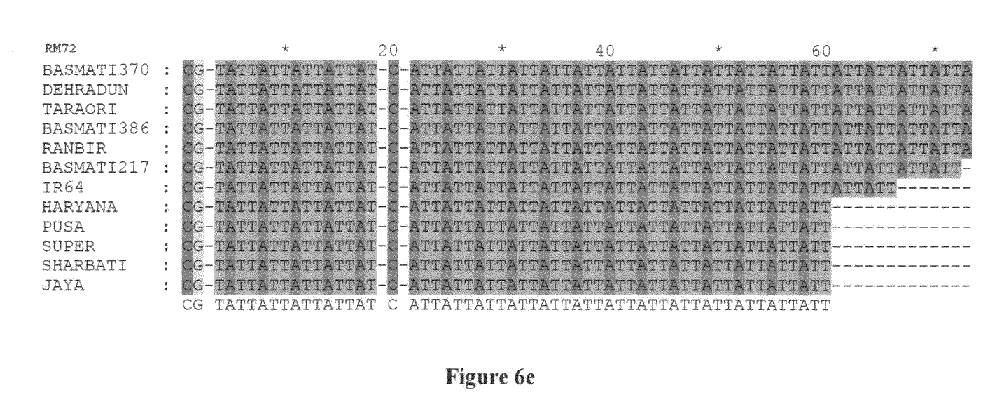

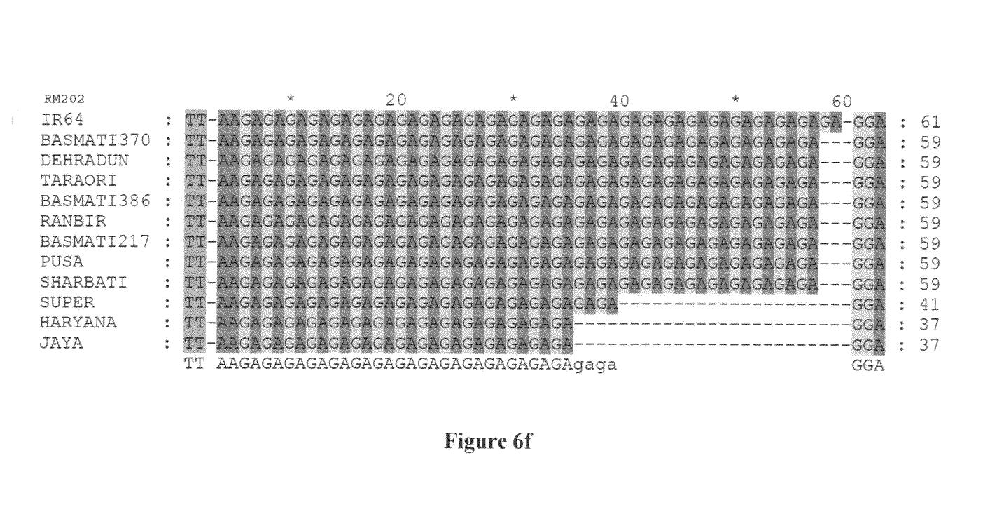

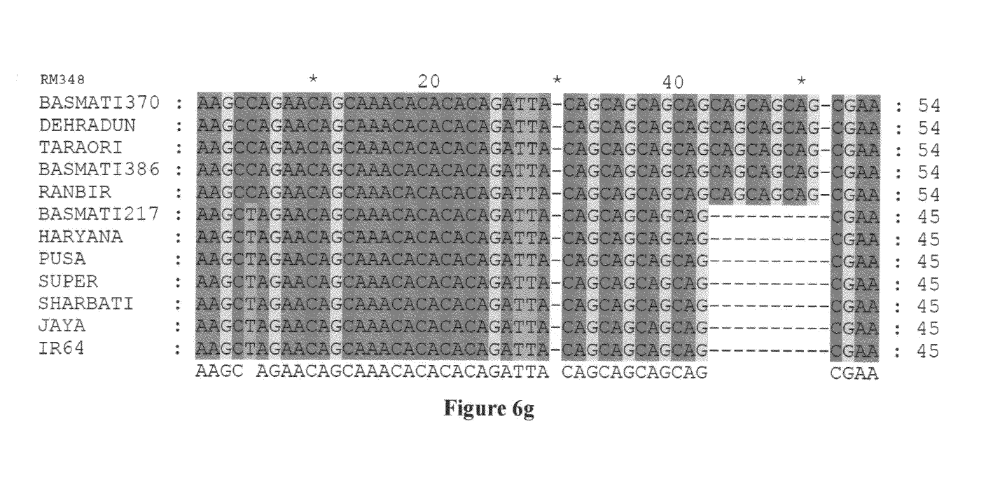

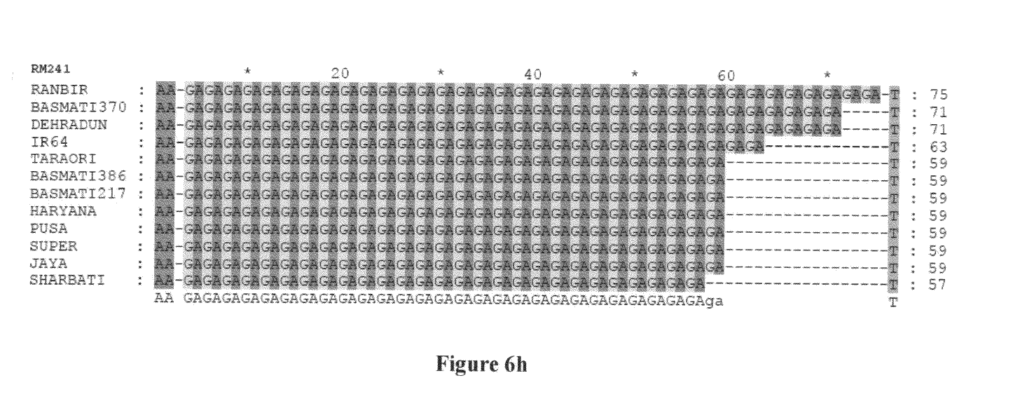

Bi-directional sequencing of PCR products was carried out thrice on ABI 3100 sequencer using ABI PRISM BigDye Primer Cycle Sequencing Kit according to the manufacturer's instructions. Sequence alignment data of various loci (RM44, RM1, RM171, RM72, RM292, RM348 and RM241), similar to the alignment sequence for locus RM55 in FIG. 2 is shown in FIGS. 6b-6h, respectively.

The invention is further elaborated with the help of following examples. However the examples should not be construed to limit the scope of the invention.

### Example 1

### Multiplex PCR

PCR amplification was carried with the following reaction mixture composition. 10 ng of DNA template, 80 μM dNTPs, 2 mM MgCl₂, primer-mix providing 0.1 μM of each primer pair to the reaction, 0.5 unit Ampli Taq Gold DNA polymerase (Applied Biosystems), were mixed in a reaction volume of 10 μl. 5′ ends of forward primers were labelled with any one of the following fluorescent ligands: TAMRA, JOE or FAM (Sigma). After an initial denaturation of 15 min at 95° C., the PCR mix was cycled 30 times at 94°, 55° and 72° C. for 30, 90 and 60 seconds respectively. This was followed by a final extension step at 60° C. at 30 min. Amplification was carried out on a PE9700 thermal cycler.

### Example 2

### Genotyping

Amplification was confirmed on 1.5% agarose gel before running genotyping assays on the capillary-based ABI 3100 genetic analyser according to manufacturer's instructions. 0.2 μl PCR product was mixed with ROX-500 size standard and Hi-dye before loading. Subsequent to electrophoresis, lanes were extracted and analysed using GeneScan version 3.1 and allele sizes of the true peaks were determined by Genotyper version 2.1. Bi-directional sequencing of PCR products was carried out thrice on ABI 3100 sequencer to obtain accurate sequences of the repeat regions.

### Example 3

### Quantification of Adulterant

Standard curves were constructed for a combination of Basmati370:Sharbati mixtures at two discriminating loci, RM72 and RM348. Standard samples were prepared by mixing the grains of the Basmati370 with Sharbati at progressive ratio of 1, 3, 5, 7, 10, 15, 17, 20, 25, 30, 40 and 60% to generate data at 12 score points. Triplicate 1 g samples at each score point were used for DNA isolation. Subsequent to genotyping, peak areas were determined for each score point and were plotted against the percent adulterant to develop a standard curve based on logistic model (y=a/l+be^{-cx}). A standard curve was also generated by mixing DNA isolated from the milled grains of Sharbati in various ratio at 5%, 10%, 20%, 30%, 40%, 50% and 60% to Basmati370 DNA to generate seven score points on the curve. Systematic bias associated with the employment of standard curves was calculated. The differences were averaged over three independent runs to compute the bias (b) at each score point. Bias (B) introduced by using standard curve was computed as, B=√Σb².

### Results

1. Variety Specific Profiles and Identification

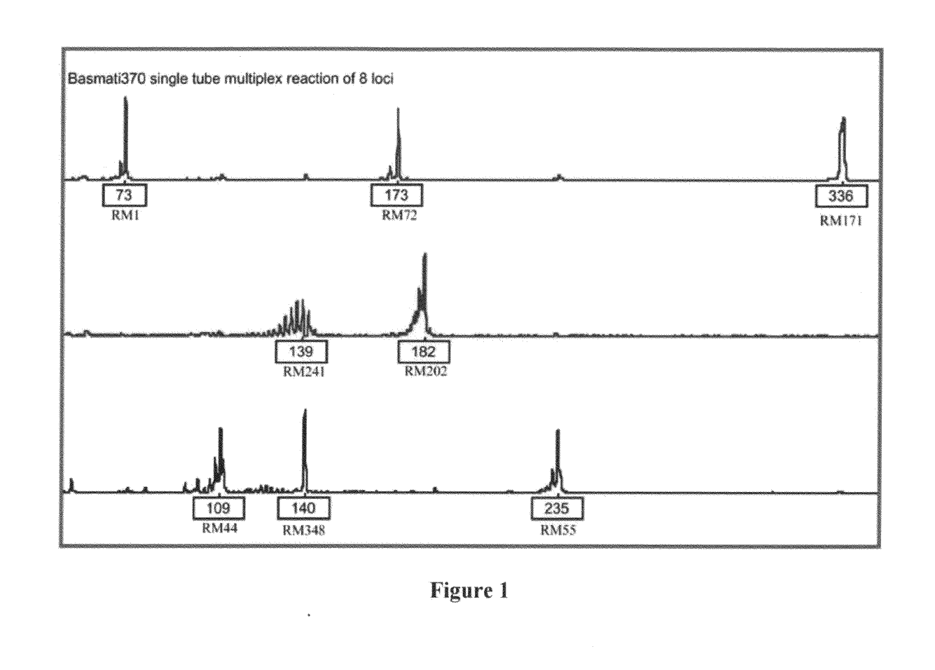

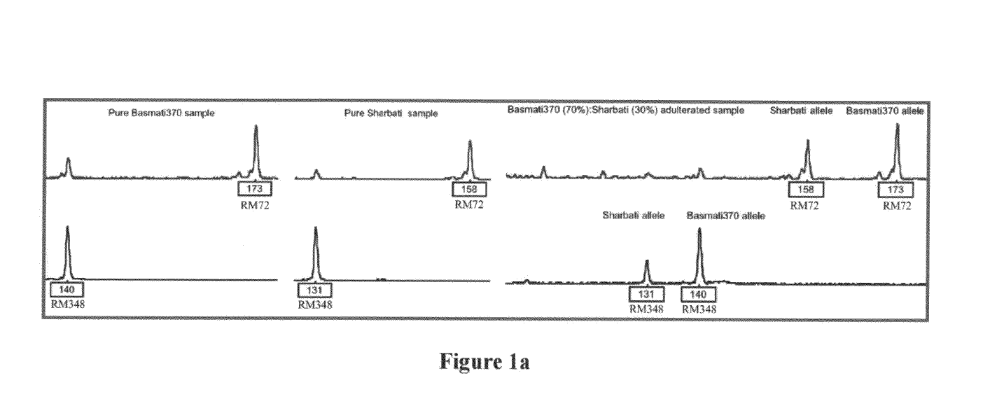

Excellent quality peaks were obtained in the single assay multiplex reactions to obtain allele sizes for all the rice varieties tested (Table 3). FIG. 1 shows the multiplex profile (8 loci) for Basmati370, FIG. 1a shows the allele profile (2 loci) of pure and adulterated Basmati370 samples. All varieties were assigned specific profiles (Table 4). The multiplex single assay can identify all the listed basmati varieties. RM171 alone can clearly separate traditional basmati from others.

Confirmation of Allele Sizes

Microsatellite alleles may produce stutters even under best of the conditions. Determination of the allele sizes can therefore be prone to errors, which is not acceptable for sensitive assays such as determination of adulterants. We confirmed the allele sizes in twelve varieties by Bi-directional sequencing of the alleles and actual counting the number of repeat units in each allele at all the loci. Sequencing also helps discover reasons for the size differences between alleles. Sequencing of PCR products was carried out thrice on ABI 3100 sequencer. In RM55, the size differences between alleles were due to disparate repeat numbers as well as indel events in the flanking sequences (FIG. 2). In all other loci, differences in the allele sizes were entirely due to differences in the number of repeat units. We therefore have confirmed sizes of all the alleles at all loci.

Quantification of the Adulterant

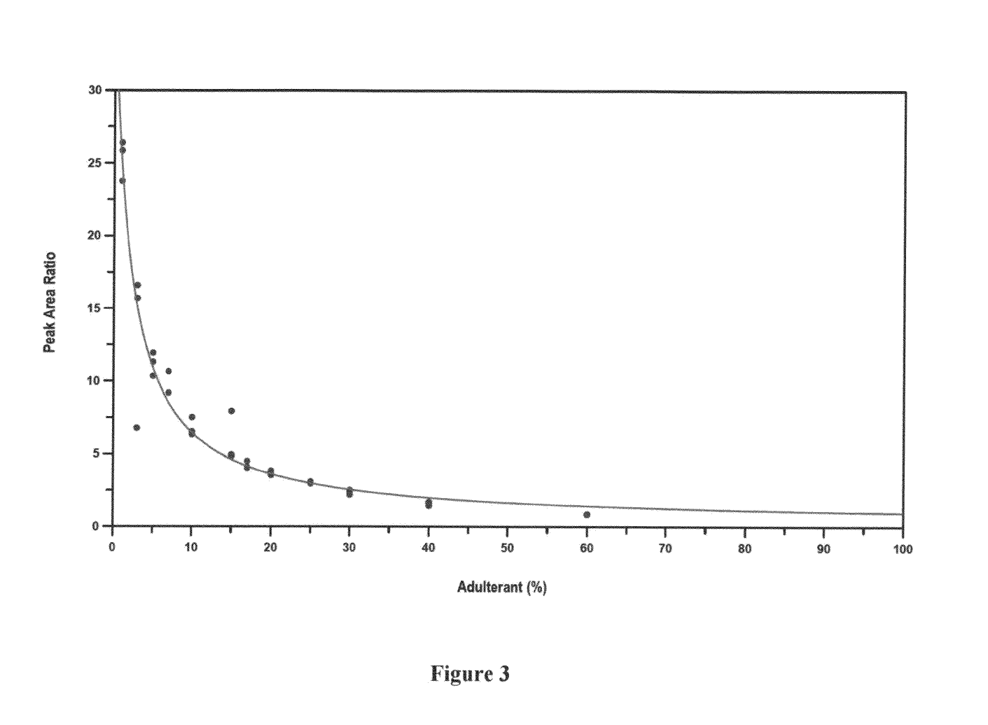

Sample standard curve obtained at RM348 is shown in FIG. 3. Systematic bias associated with the employment of standard curves was calculated to be ±4.95% for RM72 based curve and ±5.2% for RM348, based curve in the region of 1-15% adulteration. The standard curves were validated by quantifying the adulteration in blind samples. Three blind samples with 4%, 8% and 12% adulteration were genotyped and the peak-area ratios were plotted on the standard curves. The percent adulteration was estimated with an error of ±2.6% and ±2.3% respectively for RM348 and RM72 based curves. Therefore our protocol quantifies the adulterant with an accuracy of at least ±3% adulteration.

TABLE 1
List of varieties used for standardisation of multiplex

| | | Non-Basmati |
|---|---|---|
| Traditional Basmati | Evolved Basmati | long-grain |
| Varieties | varieties | varieties |
| Basmati 370 | Haryana Basmati | Sharbati |
| Type-3 (Dehradun) | Pusa basmati | IR-64 |
| Taraori basmati (HBC-I9) | Super basmati | |
| Basmati 386 | Basmati385 | |
| Ranbir basmati | | |
| Basmati 217 | | |

TABLE 2
SSR loci that are selected to distinguish basmati
from non-basmati subsequent to large-scale
screening. Loci marked with asterisk are added
in the CIP.

| | Locus | Repeat Motif | |
|---|---|---|---|
| 1. | RM 1 | (AG)₂₆ | SEQ ID NO: 1 |
| 2. | RM 110 | (GA)₁₅ | SEQ ID NO: 2 |
| 3. | RM 171 | (GATG)₅ | SEQ ID NO: 3 |
| 4. | RM 201 | (GA)₁₇ | SEQ ID NO: 4 |
| 5. | RM 202* | (GA)₃₀ | SEQ ID NO: 5 |
| 6. | RM 212 | (GA)₂₄ | SEQ ID NO: 6 |
| 7. | RM 241* | (GA)₃₁ | SEQ ID NO: 7 |
| 8. | RM 263 | (GA)₃₄ | SEQ ID NO: 8 |
| 9. | RM 282 | (GA)₁₅ | SEQ ID NO: 9 |
| 10. | RM 339 | (CTT)₈CCT(CTT)₅ | SEQ ID NO: 10 |
| 11. | RM 348* | (CAG)₇ | SEQ ID NO: 11 |
| 12. | RM 44* | (GA)₁₆ | SEQ ID NO: 12 |
| 13. | RM 440* | (CTT)₂₂ | SEQ ID NO: 13 |
| 14. | RM 525* | (AAG)₁₂ | SEQ ID NO: 14 |
| 15. | RM 55* | (GA)₁₇ | SEQ ID NO: 15 |
| 16. | RM 72 | (TAT)₅C(ATT)₁₅ | SEQ ID NO: 16 |

TABLE 3
The panel of ten informative SSR loci selected multiplex assay

| | | Chromosome | | |
|---|---|---|---|---|
| Locus | Repeat motif | no. | Forward primer | Reverse primer |
| RM171 | (GATG)₅ | 10 | AACGCGAGGACACGTACTTAC | ACGAGATACGTACGCCTTTG |
| RM55 | (GA)₁₇ | 3 | CCGTCGCCGTAGTAGAGAAG | TCCCGGTTATTTTAAGGCG |
| RM202 | (GA)₃₀ | 11 | CAGATTGGAGATGAAGTCCTCC | CCAGCAAGCATGTCAATGTA |
| RM72 | (TAT)₅C(ATT)₁₅ | 8 | CCGGCGATAAAACAATGAG | GCATCGGTCCTAACTAAGGG |
| RM348 | (CAG)₇ | 4 | CCGCTACTAATAGCAGAGAG | GGAGCTTTGTTCTTGCGAAC |
| RM241 | (GA)₃₁ | 4 | GAGCCAAATAAGATCGCTGA | TGCAAGCAGCAGATTTAGTG |
| RM44 | (GA)₁₆ | 8 | ACGGGCAATCCGAACAACC | TCGGGAAAACCTACCCTACC |
| RM1 | (AG)₂₆ | 1 | GCGAAAACACAATGCAAAAA | GCGTTGGTTGGACCTGAC |
| RM440 | (CTT)₂₂ | 5 | CATGCAACAACGTCACCTTC | ATGGTTGGTAGGCACCAAAG |
| RM525 | (AAG)₁₂ | 2 | GGCCCGTCCAAGAAATATTG | CGGTGAGACAGAATCCTTACG |

Repeat motif column discloses, from top to bottom, SEQ ID NOS: 3, 15, 5, 16, 11, 7, 12, 1, 13 and 14. Forward primer column discloses, from top to bottom, 18-27. Reverse primer column discloses, from top to bottom, SEQ ID NOS 28-37.

TABLE 4
Allele sizes (in base pairs) of various basmati rice varieties obtained
by multiplex single assay method

| | | Dehradun | Taraori | | Ranbir | | Haryana | Pusa | Super | | | |
|---|---|---|---|---|---|---|---|---|---|---|---|---|
| Locus | Basmati370 | Basmati | Basmati | Basmati386 | Basmati | Basmati217 | Basmati | Basmati | Basmati | Basmati385 | Sharbati | IR64 |
| RM 1 | 73 | 73 | 73 | 73 | 73 | 100 | 108 | 73 | 106 | 73 | 106 | 106 |
| RM72 | 173 | 173 | 173 | 173 | 173 | 158 | 158 | 158 | 158 | 158 | 158 | 164 |
| RM171 | 335 | 335 | 335 | 335 | 335 | 343 | 343 | 343 | 343 | 335 | 322, 343, 335 | 346 |
| RM241 | 140 | 140 | 128 | 128 | 144 | 128 | 128 | 128 | 128 | 140 | 128 | 128 |
| RM202 | 182 | 182 | 182 | 182 | 182 | 182 | 161 | 182 | 164 | 161 | 161 | 186 |
| RM44 | 109 | 109 | 113 | 113 | 109 | 103 | 103 | 113 | 103 | 113 | 103 | 103 |
| RM348 | 139 | 139 | 139 | 139 | 139 | 130 | 130 | 130 | 130 | 230 | 130 | 130 |
| RM55 | 235 | 235 | 219 | 219 | 235 | 230 | 230 | 230 | 230 | 139 | 230 | 230 |
| RM440 | 150 | 146 | 150 | 150 | 146 | 146 | 150 | 202 | 150, | 150, | 150 | 202 |
| | | | | | | | | | 202 | 202 | | |
| RM525 | 146 | 146 | 146 | 146 | 146 | 106 | 146 | 146 | 146 | 106 | 106 | 106 |

TABLE 5
Genotype codes of various basmati rice varieties based on single
assay multiplex method. The order of codes from left to right correspond to
loci 1 to 8 given in Table 5. Shaded part to show traditional basmati
varieties.

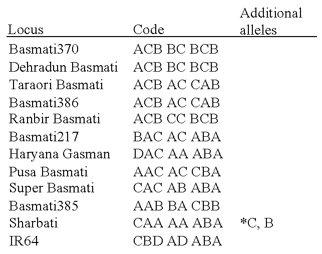

TABLE 6
Allele sizes in base pairs for corresponding codes of Table 4.

| # | Locus | A | B | C | D |
|---|---|---|---|---|---|
| 1. | RM1 | 73 | 100 | 106 | 108 |
| 2. | RM72 | 158 | 164 | 173 | |
| 3. | RM171 | 322 | 335 | 343 | 346 |
| 4. | RM241 | 128 | 140 | 144 | |
| 5. | RM202 | 161 | 164 | 182 | 186 |
| 6. | RM44 | 103 | 109 | 113 | |
| 7. | RM55 | 219 | 230 | 235 | |
| 8. | RM348 | 130 | 139 | | |

TABLE 7
Arrangement of 10 primers in a particular manner to facilitate
single genotyping assay. It is clear from the table that
loci were grouped so as to avoid overlapping allele sizes
in the same fluorescence label (read as ‘same coloured
peaks in the electrophoresis’) as shown in FIG. 7.

| | Allele pool | |
|---|---|---|
| | (in base pairs) | Fluorophore |
| Locus | | |
| RM1 | 73, 100, 106, 108 | FAM |
| RM72 | 158, 164, 173 | FAM |
| RM171 | 322, 335, 343, 346 | FAM |
| RM202 | 161, 164, 182, 186 | JOE |
| RM241 | 128, 140, 144 | JOE |
| RM44 | 103, 109, 113 | TAMRA |
| RM55 | 219, 230, 235 | TAMRA |
| RM348 | 130, 139 | TAMRA |
| Additional Loci | | |
| RM440 | 146, 150, 202 | As needed |
| RM525 | 106, 146 | As needed |

1. (canceled)

2. A single tube multiplex assay for distinguishing rice varieties in a sample, wherein said varieties are selected from a class of rice comprising traditional basmati, evolved basmati, non-basmati or any combination thereof, said assay comprising steps of:
a) amplifying a combination of at least 8 loci selected from a group comprising RM1, RM55, RM202, RM348, RM241, RM44, RM440, RM525, RM171 and RM72 of DNA from a sample comprising basmati or non-basmati rice varieties or a combination of rice varieties in a single tube using forward primers having SEQ ID NO.: 18 to SEQ ID NO.: 27 respectively, and reverse primers having SEQ ID NO.: 28 to SEQ ID NO.: 37 respectively, wherein the DNA is in an amount of 10 ng/10 μl;
b) carrying out electrophoresis of the amplified product to identify alleles and the corresponding allele peaks at said loci and determining sizes of the allele peaks; and
c) comparing the allele peaks with standard allelic profile peaks of basmati for said loci for determining a ratio of the allele peaks to distinguish the rice varieties.
